(19) United States
(12) Patent Application Publication
(10) Pub. No.: US 2024/0207628 A1
(43) Pub. Date: Jun. 27, 2024

(54) Systems And Method For Array Positioning

(71) Applicant: NOVOCURE GMBH, Root D4 (CH)

(72) Inventor: Kathryn Smith-Petersen, Portsmouth, NH (US)

(21) Appl. No.: 18/392,835

(22) Filed: Dec. 21, 2023

Related U.S. Application Data
(60) Provisional application No. 63/477,012, filed on Dec. 23, 2022.

(51) Int. Cl. A61N 1/40; A61B 90/00

(52) U.S. Cl. CPC A61N 1/40; A61B 90/39; A61B 2090/395

(57) ABSTRACT

A system comprises an electrode array comprising a plurality of electrodes and a marker configured to be associated with skin of a patient for indicating a location on the skin for positioning the electrode array. The system can assist the patient in positioning the electrode array, particularly in areas where directly viewing the location is difficult.

### CROSS-REFERENCE TO RELATED APPLICATION

This application claims priority to and the benefit of the filing date of U.S. Provisional Patent Application No. 63/477,012, filed Dec. 23, 2022, the entirety of which is hereby incorporated by reference herein.

### FIELD

This application relates to systems and methods for positioning electrode arrays.

### BACKGROUND

Tumor Treating Fields (TTFields) therapy is a proven approach for treating tumors using alternating electric fields at frequencies between 50 kHz and 1 MHz and, more commonly, between 100 kHz and 500 kHz. In current commercial systems, the alternating electric fields are induced by electrode assemblies (e.g., arrays of capacitively coupled electrodes, also called transducer arrays or electrode arrays) placed on opposite sides of a target region of the subject's body. When an AC voltage is applied between opposing electrode arrays, an AC current is coupled through the electrode arrays and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

### SUMMARY

TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a combination therapy with chemotherapy for malignant pleural mesothelioma (MPM), and may find use in treating tumors in other parts of the body. For applications targeting tumors in the torso, larger electrode arrays than currently used with the OPTUNE® system may be beneficial.

Disclosed herein, in one aspect, is a system comprising an electrode array comprising a plurality of electrodes and a marker configured to be associated with skin of a patient for indicating a location on the skin for positioning the electrode array.

In one aspect, an electrode array comprises a plurality of electrodes; and a marker sensor that is configured to detect proximity of a marker. In some aspects, a system comprises the electrode array and an indicator in communication with the marker sensor, wherein the indicator is configured to cause an indication upon the marker sensor being in proximity of the marker.

In one aspect, a method comprises positioning an electrode array comprising a plurality of electrodes onto skin of a patient based on a marker associated with the skin of the patient, wherein the marker indicates a location on the skin for positioning the electrode array.

In one aspect, a method comprises orienting an electrode array on a patient, wherein the electrode array comprises a marker sensor, wherein orienting the electrode array comprises positioning the electrode array based on feedback from an indicator in communication with the marker sensor.

In one aspect, a method comprises implanting a marker under the skin of the patient, wherein the marker is indicative of a location for positioning an electrode array for providing tumor treating fields.

### BRIEF DESCRIPTION OF THE DRAWINGS

FIG. 1 is a schematic view of a system for delivering tumor treating fields as disclosed herein.

FIG. 2 is a schematic view of an exemplary system for orienting an electrode array in accordance with embodiments disclosed herein.

FIG. 3 is a schematic view of an exemplary system for orienting an electrode array in accordance with embodiments disclosed herein.

FIG. 4 is a schematic view of an exemplary system for orienting an electrode array in accordance with embodiments disclosed herein.

FIG. 5 is a block diagram of an operating environment comprising a computing device of the system of FIG. 2.

Various embodiments are described in detail below with reference to the accompanying drawings, wherein like reference numerals represent like elements, and wherein descriptions of like elements may not be repeated for every embodiment, but may be considered to be the same if previously described herein.

### DESCRIPTION OF THE PREFERRED EMBODIMENTS

This application relates to positioning of electrode arrays that may be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an,” and “the” can optionally include plural referents unless the context clearly dictates otherwise.

### System for Providing Tumor Treating Fields

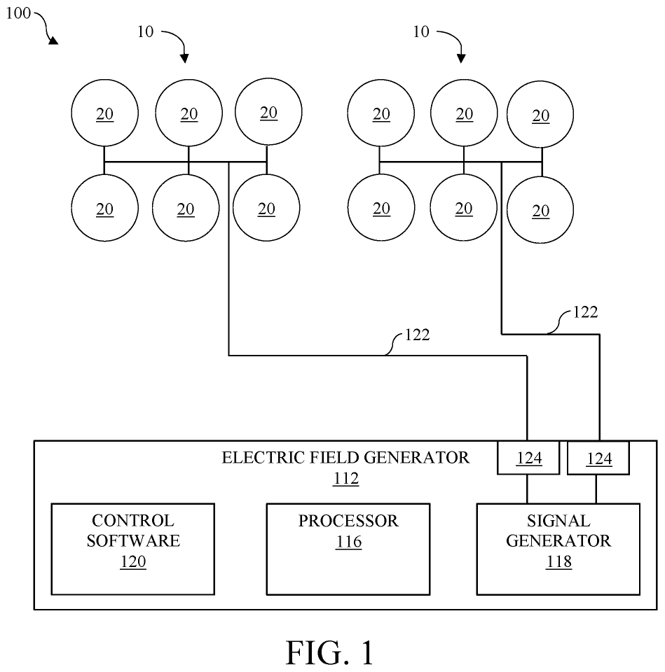

FIG. 1 shows an example apparatus 100 for electrotherapeutic treatment. Generally, the apparatus 100 may be a portable battery or power supply operated device that produces alternating electrical fields within the body by means of transducer arrays or other electrodes. The apparatus 100 may comprise an electrical field generator 112 and one or more electrode (e.g., transducer) arrays (e.g., assemblies 10), each comprising a plurality of electrodes 20. The apparatus 100 may be configured to generate tumor treating fields (TTFields), for example, at frequencies in the range of from about 50 kHz to about 1 MHz, such as from 50 kHz to about 500 kHz (e.g., at about 150 kHz for one tumor cell type, and/or at about 300 kHz for a different tumor cell type), via the electrical field generator 112, and deliver the TTFields to an area of the body through the one or more electrode arrays 10. The electrical field generator 112 may be a battery and/or power supply operated device.

The electrical field generator 112 may comprise a processor 116 in communication with a signal generator 118. The electrical field generator 112 may comprise control software 120 (controller) configured for controlling the operation of the processor 116 and the signal generator 118. Although depicted as a single processor 116, it is contemplated that a plurality of processors can be provided.

The signal generator 118 may generate one or more electric signals in the shape of waveforms or trains of pulses. The signal generator 118 may be configured to generate an alternating voltage waveform, for example, at frequencies in the range from about 50 kHz to about 500 kHz (preferably from about 100 kHz to about 300 kHz) (e.g., the TTFields). The voltages are such that the electrical field intensity in tissue to be treated is typically in the range of about 0.1 V/cm to about 10 V/cm.

One or more outputs 124 of the electrical field generator 112 may be coupled to one or more conductive leads 122 that are attached at one end thereof to the signal generator 118. The opposite ends of the conductive leads 122 are connected to the one or more electrode arrays 10 that are activated by the electric signals (e.g., waveforms). The conductive leads 122 may comprise standard isolated conductors with a flexible metal shield and may be grounded to prevent the spread of the electrical field generated by the conductive leads 122. The one or more outputs 124 may be operated sequentially. Output parameters of the signal generator 118 may comprise, for example, an intensity of the field, a frequency of the waves (e.g., treatment frequency), and a maximum allowable temperature of the one or more electrode arrays 10. The output parameters may be set and/or determined by the control software 120 (controller) in conjunction with the processor 116. After determining a desired (e.g., optimal) treatment frequency, the control software 120 may cause the processor 116 to send a control signal to the signal generator 118 that causes the signal generator 118 to output the desired treatment frequency to the one or more electrode arrays 10. It is further contemplated that the control software 120 can cause the processor 116 to shift or change the direction of TTFields or otherwise adjust the properties of TTFields in the manner further disclosed herein.

### System for Positioning Electrode Array

Disclosed herein is a system for assisting a patient in positioning the electrode array in a particular location on the skin of the patient, particularly in areas where directly viewing the location is difficult.

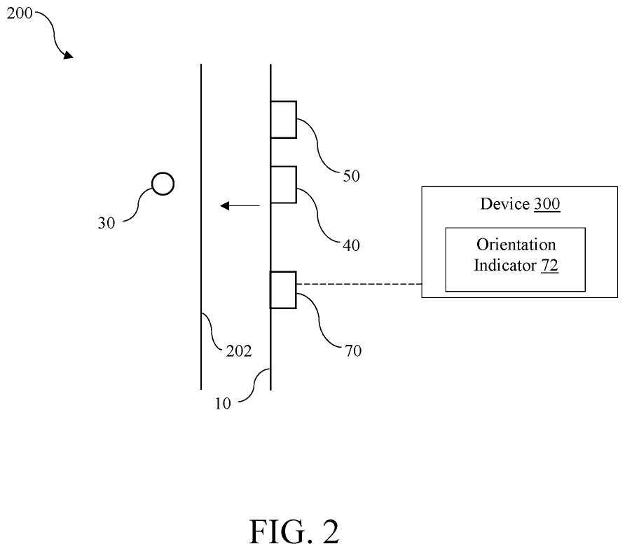

Referring to FIG. 2, the system 200 can comprise an electrode array 10 (also referred to herein as “the array”) comprising a plurality of electrodes 20 (FIG. 1). The system 200 can further comprise a marker 30 that is configured to be associated with skin 202 of a patient for indicating a location on the skin for positioning the electrode array.

In some aspects, the marker 30 can be configured to be implanted under the skin of the patient.

The marker 30 can comprise a material that is configured to guide an electric field. For example, in some aspects, the material can be conductive. In other aspects, the material can be dielectric.

The marker 30 can comprise a biocompatible material. For example, in some aspects, the biocompatible material can be, or can comprise, titanium.

In some aspects, the electrode array 10 can comprise a marker sensor 40 that is configured to detect proximity of the marker 30. The marker sensor 40 can be, for example, an electromagnetic sensor. The system 200 can further comprise an indicator 50 in communication with the marker sensor 40. The indicator 50 can be configured to cause an indication upon the marker sensor 40 being in proximity of the marker 30. In further aspects, the marker sensor 40 can be configured to detect alignment between the marker sensor 40 and the marker 30. In these aspects, the indicator 50 can be configured to cause the indication upon the marker sensor 40 being in alignment with the marker 30. The electrode array 10 being in proximity of and/or alignment with the marker 30 can correspond to the electrode array being correctly positioned on the patient. In some optional aspects, a detected proximity of the marker can correspond to a location that is in alignment with the marker. In other optional aspects, the indicator can be configured to indicate both proximity and alignment. For example, in some aspects, the marker 30 and marker sensor 40 can cooperate to indicate alignment. In some aspects, the marker 30 can be able to cooperate with the marker sensor 40 to generate an electromagnetic signal based on rotational alignment of the electrode array 10 relative to the marker. Optionally, in these aspects, the marker can be elongate so that opposed ends of the marker can each be indicative of a respective position. Thus, for example, the electrode array 10 can be oriented relative to both ends of the marker. In further aspects, the marker can comprise a plurality of elements that are positioned relative to each other to cooperate with the marker sensor 40 to produce a signal indicative of alignment.

In some aspects, the indicator 50 can comprise an audible indicator, a visual indicator, or a haptic indicator, or combinations thereof. In some aspects, the indicator 50 can comprise a speaker. In some aspects, the indicator can comprise an LED or other light emitting device. In some aspects, the indicator 50 can comprise a vibrating indicator. In some aspects, the indicator 50 can be configured to provide a binary indication as to whether the electrode array is properly positioned (e.g., yes/no). For example, the indicator can comprise a light that flashes or outputs a particular color (e.g., red) when the electrode array 10 is outside of a threshold, and stays constantly lit or outputs a particular color (e.g., green) when the electrode array 10 is within the threshold. Said threshold can be a spacing between the marker 30 and the marker sensor 40. In other aspects, the indicator can be configured to provide a more continuous output (e.g., beeping, flashing, or vibrating at a rate depending on proximity of proper positioning).

In some aspects, the indicator 50 can be coupled to the electrode array 10. For example, in some aspects, the indicator 50 can be integral to the electrode array 10. In some aspects, the indicator 50 can be associated with a device 300 that is separate from the electrode array. For example, the device 300 can be a smartphone, smartwatch, wearable electronic device, or other stand-alone computing device. Accordingly, in some aspects, the device 300 can be in communication with the marker sensor. The device 300 can execute an application that causes the device to output, on a display of the device, the indication caused by the indicator. The indication can be binary (e.g., yes/no). In other aspects, the indication can provide greater resolution (e.g., showing a number, color, plot, or other output indicating relative proximity of the marker sensor 40 to the marker 30). In some optional aspects, it is contemplated that the displayed indication can change in real-time in response to changes in the relative positions of the marker 30 and the marker sensor 40.

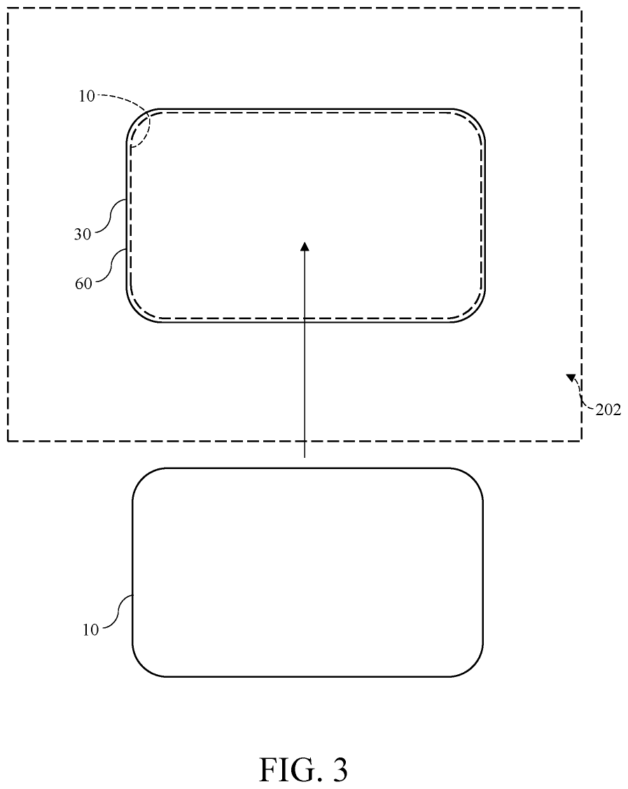

Referring to FIG. 3, in some optional aspects, the marker 30 can comprise a visible marker. For example, in some aspects, the visible marker can comprise a marking 60 such as a temporary tattoo for positioning on the skin 202. The temporary tattoo can optionally be a henna tattoo. In some aspects, the visible marker can comprise glow in the dark ink or black light ink.

Optionally, the visible marker can comprise indications of at least a portion of a perimeter of the electrode array. For example, the visible marker can include a trace of an outline, or a portion of an outline, of the electrode array 10.

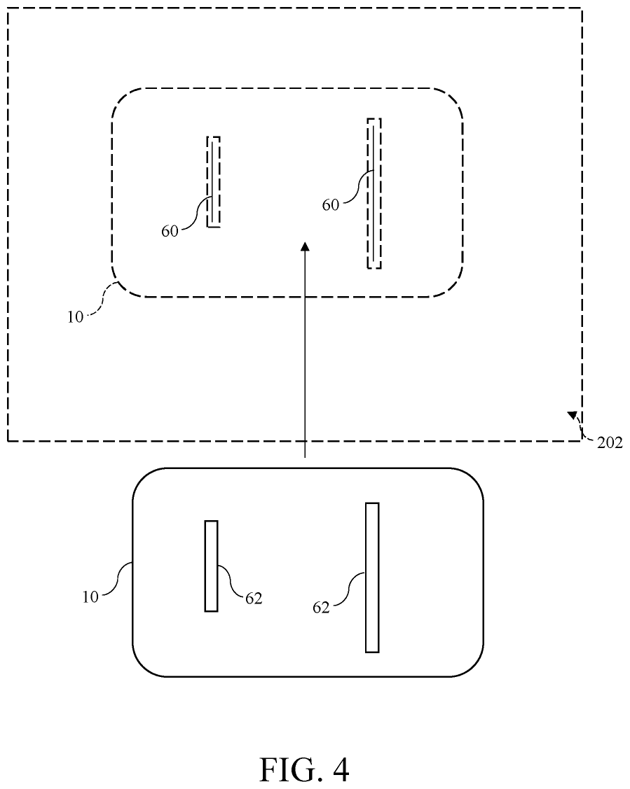

Referring to FIG. 4, in some aspects, the electrode array 10 can define at least one opening 62 therethrough. The visible marker can comprise at least one marking 60 that is visible through the at least one opening when the electrode array is in the location on the skin 202 for positioning the electrode array.

Optionally, electrode array 10 can define a plurality of openings 62 therethrough. The at least one marking 60 can comprise a plurality of markings 60 that are visible through respective openings 62 of the plurality of openings 62 when the electrode array is in the location on the skin for positioning the electrode array.

For example, in some aspects, the plurality of openings 62 can comprise a plurality of slots. Optionally, as shown in FIG. 4, in some aspects, the plurality of markings 60 can comprise a plurality of elongate markings that are visible through respective slots 62 of the plurality of slots when the electrode array 10 is in the location on the skin for positioning the electrode array.

Referring to FIG. 2, the electrode array 10 can comprise an orientation sensor 70 that is configured to detect an orientation of the electrode array 10. In some optional aspects, the orientation sensor 70 can comprise an accelerometer. The orientation sensor 70 can be in communication with an orientation indicator 72. The orientation indicator 72 can be configured to cause an indication of the orientation sensed by the orientation sensor. In some aspects, the orientation indicator 72 can comprise an audible indicator (e.g., comprising a speaker), a visual indicator (e.g., comprising a display or light), a haptic indicator (e.g., causing a vibration), or combinations thereof.

In some aspects, the orientation indicator 72 can be coupled to the electrode array 10. For example, in some aspects, the orientation indicator can be integral to the electrode array 10. In some aspects, the orientation indicator 72 can be associated with a device 300 that is separate from the electrode array 10. For example, the device 300 can be a smartphone, smartwatch, wearable electronic device, or other stand-alone computing device. Accordingly, in some aspects, the device 300 can be in communication with the orientation sensor 70. The device 300 can execute an application that causes the device to output, on a display of the device, the indication of the orientation. The indication can be binary (e.g., incorrectly oriented or properly oriented, yes/no, etc.). In other aspects, the indication can provide greater resolution (e.g., showing a numerical angular offset, color, plot of angular offset, or other output indicating orientation relative to a desired orientation of the electrode array).

In some aspects, the orientation sensor 70 can comprise a camera of the device 300. The camera can detect an orientation of the electrode array (e.g., by detecting an orientation of a line or other marking on the electrode array). The camera can further detect a proper orientation based on the at least one visible marker (e.g., by detecting the at least one visible marker on the skin of the patient). For example, the camera can acquire an image that is then processed (e.g., by a processor of the device 300 in communication with the camera) to make determinations of placement and/or orientation of the electrode array 10. In some aspects, the camera can detect the presence or absence of the at least one visible marker 60 through the at least one opening 62 through the electrode array 10 (FIG. 4). The processor in communication with the camera can determine, based on the presence or absence of the at least one visible marker within (and, optionally, properly oriented within) the at least one opening.

In some aspects, the device 300 can compare the orientation of the device to the proper orientation to provide an output to the patient. The indication can be binary (e.g., incorrectly oriented or properly oriented, yes/no, etc.). For example, in some aspects, the indication can be provided based on whether the orientation is inside or outside of a threshold (e.g., optionally, +/−5 degrees). In other aspects, the indication can provide greater resolution (e.g., showing a numerical angular offset, color, plot of angular offset, or other output indicating orientation relative to a desired orientation of the electrode array).

### Method of Positioning an Electrode Array

A method can comprise positioning an electrode array 10 comprising a plurality of electrodes 20 onto skin of a patient based on a marker 30 associated with the skin of the patient. The marker 30 can be associated with the skin of the patient for indicating a location on the skin for positioning the electrode array.

In some aspects the marker 30 can be implanted under the skin of the patient.

Optionally, the marker 30 can comprise a material that is configured to guide an electric field. For example, in some aspects, the material can be conductive. In other aspects, the material can be dielectric.

The marker 30 can comprise a biocompatible material. For example, in some aspects, the biocompatible material can be, or can comprise, titanium.

In some aspects, the marker can comprise a visible marker. In some optional aspects, the method can further comprise applying the marker to the skin of the patient. For example, in some aspects, the visible marker can comprise a temporary tattoo. The temporary tattoo can comprise a henna tattoo. In some aspects, the visible marker can comprise glow in the dark ink or black light ink.

A method can comprise positioning, on a patient, an electrode array 10. The electrode array 10 can comprise a marker sensor 40. Positioning the electrode array can comprise positioning the electrode array based on feedback from an indicator in communication with the marker sensor. The marker sensor 40 can be in communication with an indicator 50 that causes an indication upon the marker sensor being in proximity of the marker 30 (e.g., when the electrode array is correctly positioned on the patient). The indicator 50 can be configured to cause an indication upon the marker sensor 40 being in proximity of the marker 30. In further aspects, the marker sensor 40 can be configured to detect alignment between the marker sensor 40 and the marker 30 (e.g., when the electrode array is correctly positioned on the patient). The indicator 50 can cause the indication upon the marker sensor 40 being in alignment with the marker 30. In some optional aspects, the indicator 50 can be integral to the electrode array 10. In some optional aspects, the indicator 50 can be associated with a smartphone, smartwatch, wearable device, or other stand-alone computing device.

A method can comprise orienting, on a patient, an electrode array 10 comprising an orientation sensor 70. The electrode array 10 can be oriented based on feedback from an orientation indicator in communication with the orientation sensor. For example, the orientation indicator can provide a binary indication as disclosed herein or an indication with greater resolution, as further disclosed herein. For example, in some aspects, the indication can comprise a readout of a sensed angle relative to an optimal angle.

A method can comprise implanting a marker under the skin of the patient, wherein the marker is indicative of a location for positioning an electrode array for providing tumor treating fields.

In some aspects, the marker 30 can be implanted within a resection cavity.

In some aspects, the marker 30 can be positioned proximate to a surface of the skin such that the marker can be felt by touching the skin. In this way, the patient can feel the marker in order to determine its position and the position of the electrode array relative thereto.

In some aspects, the marker 30 can comprise a material that is configured to guide an electric field. In some aspects, the material can be conductive. In some aspects, the material can be dielectric.

In some aspects, the material can comprise a biocompatible material. Optionally, the biocompatible material can be, or comprise, titanium.

In some aspects, positioning the electrode array 10 on the skin of the patient can comprise positioning the electrode array on a back of the patient.

### Exemplary Computing Device

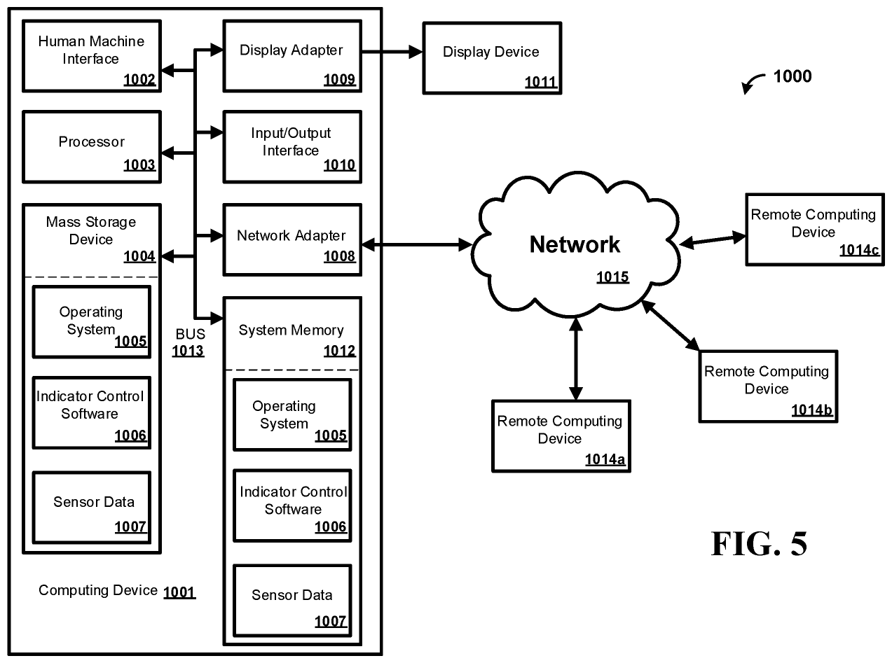

FIG. 5 shows an exemplary operating environment 1000 including an exemplary configuration of a computing device 1001 for use with the system 200 (FIG. 2) disclosed herein. For example, the computing device 1001 can be the computing device of the device 300 (FIG. 2), which can be, for example, a remote device such as a smartphone, tablet, personal computer, or other computing device configured for communication with the electrode array 10 (FIG. 2). In some aspects, the computing device 1001 can be integral to the electrode array 10 (FIG. 2). For example, the computing device 1001 can be embodied at least in part as a microcontroller that controls operation of an integral indicator. For example, the computing device 1001 (e.g., microcontroller) can be configured to receive signals from the marker sensor 40 and/or orientation sensor 70 and cause an indication by the indicator 50 and/or orientation indicator 72 based on the signals from the marker sensor 40 and/or orientation sensor 70.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as sensor data 1007 and/or program modules such as operating system 1005 and indicator control software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and indicator control software 1006 may be stored on the mass storage device 1004. One or more of the operating system 1005 and indicator control software 1006 (or some combination thereof) may comprise program modules and the indicator control software 1006. The sensor data 1007 may also be stored on the mass storage device 1004. The sensor data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/ or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display device 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. The remote computing devices 1014a,b,c, can perform respective operations of the system 200 (FIG. 2). Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the operating environment 1000 can be configured with internet connectivity.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising:
an electrode array comprising a plurality of electrodes; and
a marker configured to be associated with skin of a patient for indicating a location on the skin for positioning the electrode array.

Aspect 2: The system of aspect 1, wherein the marker is configured to be implanted under the skin of the patient.

Aspect 3: The system of aspect 2, wherein the marker comprises a material that is configured to guide an electric field.

Aspect 4: The system of aspect 3, wherein the material is conductive.

Aspect 5: The system of aspect 3, wherein the material is dielectric.

Aspect 6: The system of any one of the preceding aspects, wherein the marker comprises a biocompatible material.

Aspect 7: The system of aspect 6, wherein the biocompatible material comprises titanium.

Aspect 8: The system of any one of aspects 2-7, wherein the electrode array comprises:
a marker sensor that is configured to detect proximity of or alignment with the marker when the electrode array is correctly positioned on the patient; and
an indicator in communication with the marker sensor, wherein the indicator is configured to cause an indication upon the marker sensor being in proximity of or alignment with the marker.

Aspect 9: The system of aspect 8, wherein the indicator comprises an audible indicator, a visual indicator, or a haptic indicator.

Aspect 10: The system of aspect 9, wherein the indicator comprises a speaker.

Aspect 11: The system of aspect 9, wherein the indicator comprises an LED.

Aspect 12: The system of aspect 9, wherein the indicator comprises a vibrating indicator.

Aspect 13: The system of aspect 1, wherein the marker comprises a visible marker.

Aspect 14: The system of aspect 13, wherein the visible marker comprises a temporary tattoo.

Aspect 15: The system of aspect 14, wherein the temporary tattoo comprises a henna tattoo.

Aspect 16: The system of aspect 13, wherein the visible marker comprises glow in the dark ink or black light ink.

Aspect 17: The system of any one of aspects 13-16, wherein the visible marker comprises indications of at least a portion of a perimeter of the electrode array.

Aspect 18: The system of aspect 17, wherein the visible marker comprises an outline of at least a portion of the perimeter of each electrode array.

Aspect 19: The system of any one of aspects 13-16, wherein the electrode array defines at least one opening therethrough, wherein the visible marker comprises at least one marking that is visible through the at least one opening when the electrode array is in the location on the skin for positioning the electrode array.

Aspect 20: The system of aspect 19, wherein the at least one opening comprises a plurality of openings therethrough, wherein the at least one marking comprises a plurality of markings that are visible through respective openings of the plurality of openings when the electrode array is in the location on the skin for positioning the electrode array.

Aspect 21: The system of aspect 20, wherein the plurality of openings comprise a plurality of slots, wherein the plurality of markings comprise a plurality of elongate markings that are visible through respective slots of the plurality of slots when the electrode array is in the location on the skin for positioning the electrode array.

Aspect 22: The system of any one of the preceding aspects, wherein the electrode array comprises an orientation sensor that is configured to detect an orientation of the electrode array.

Aspect 23: The system of aspect 22, wherein the orientation sensor is in communication with an orientation indicator, wherein the orientation indicator is configured to cause an indication upon the sensor being in a proper orientation.

Aspect 24: The system of aspect 23, wherein the orientation indicator is coupled to the electrode array.

Aspect 25: The system of aspect 23, wherein the orientation indicator is associated with a device that is separate from the electrode array.

Aspect 26: The system of aspect 25, wherein the device is a smartphone, smartwatch, or wearable electronic device.

Aspect 27: The system of any one of aspects 23-26, wherein the orientation indicator comprises an audible indicator, a visual indicator, or a haptic indicator.

Aspect 28: The system of any one of aspects 23-27, wherein the orientation sensor comprises an accelerometer.

Aspect 29: An electrode array comprising:
a plurality of electrodes; and
a marker sensor that is configured to detect proximity of or alignment with a marker.

Aspect 30: The electrode array of aspect 29, further comprising an indicator in communication with the marker sensor, wherein the indicator is configured to:
cause an indication upon the marker sensor detecting proximity of or alignment with the marker.

Aspect 31: A system comprising:
the electrode array of aspect 29 or aspect 30; and
an indicator in communication with the marker sensor, wherein the indicator is configured to cause an indication upon the marker sensor being in proximity of or alignment with the marker.

Aspect 32: The system of aspect 21, wherein the indicator is associated with a smartphone.

Aspect 33: The system of any one of aspects 30-32, wherein the indication comprises a readout of a sensed angle relative to an optimal angle.

Aspect 34: A method comprising:
positioning an electrode array comprising a plurality of electrodes onto skin of a patient based on a marker associated with the skin of the patient,
wherein the marker indicates a location on the skin for positioning the electrode array.

Aspect 35: The method of aspect 34, wherein the marker is implanted under the skin of the patient, wherein the marker comprises a material that is configured to guide an electric field.

Aspect 36: The method of aspect 35, wherein the material is conductive.

Aspect 37: The method of aspect 35, wherein the material is dielectric.

Aspect 38: The method of any one of aspects 34-37, wherein the marker comprises a biocompatible material.

Aspect 39: The method of aspect 38, wherein the biocompatible material comprises titanium.

Aspect 40: The method of any one of aspects 35-39, wherein positioning the electrode array based on the marker associated with the skin of the patient comprises feeling the position of the marker under the skin.

Aspect 41: The method of any one of aspects 34-40, wherein the electrode array comprises:
a marker sensor that is configured to detect proximity of or alignment with the marker; and
an indicator in communication with the marker sensor, wherein the indicator is configured to cause an indication upon the marker sensor being in proximity of or alignment with the marker, wherein positioning the electrode array comprises positioning the electrode array based on the indication from the indicator.

Aspect 42: The method of aspect 41, wherein the indicator comprises an audible indicator, a visual indicator, or a haptic indicator.

Aspect 43: The method of aspect 42, wherein the indicator comprises a speaker.

Aspect 44: The method of aspect 42, wherein the indicator comprises an LED.

Aspect 45: The method of aspect 42, wherein the indicator comprises a vibrating indicator.

Aspect 46: The method of aspect 34, wherein the marker comprises a visible marker.

Aspect 47: The method of aspect 46, wherein the visible marker comprises a temporary tattoo.

Aspect 48: The method of aspect 47, wherein the temporary tattoo comprises a henna tattoo.

Aspect 49: The method of aspect 48, wherein the visible marker comprises glow in the dark ink or black light ink.

Aspect 50: The method of any one of aspects 46-50, wherein the visible marker comprises indications of at least a portion of a perimeter of the electrode array.

Aspect 51: The method of aspect 50, wherein the visible marker comprises an outline of at least a portion of the perimeter of each electrode array.

Aspect 52: The method of any one of aspects 46-51, wherein the electrode array defines at least one opening therethrough, wherein the visible marker comprises at least one marking that is visible through the at least one opening when the electrode array is in the location.

Aspect 53: The method of aspect 52, wherein the at least one opening comprises a plurality of openings therethrough, wherein the at least one marking comprises a plurality of markings that are visible through respective openings of the plurality of openings when the electrode array is in the location.

Aspect 54: The method of aspect 53, wherein the plurality of openings comprise a plurality of slots, wherein the plurality of markings comprise a plurality of elongate markings that are visible through respective slots of the plurality of slots when the electrode array is in the location.

Aspect 55: A method comprising:
orienting an electrode array on a patient, wherein the electrode array comprises a marker sensor, wherein orienting the electrode array comprises positioning the electrode array based on feedback from an indicator in communication with the marker sensor.

Aspect 56: The method of aspect 55, wherein the marker sensor is in communication with an indicator that causes an indication upon the marker sensor being in proximity of or alignment with the marker.

Aspect 57: The method of aspect 56, wherein the indicator is configured to cause an indication upon the marker sensor being in proximity of or alignment with the marker.

Aspect 58: The method of aspect 57, wherein the indicator is associated with a smartphone, smartwatch or wearable device.

Aspect 59: The method of any one of aspects 55-58, wherein the indication comprises a readout of a sensed angle relative to an optimal angle.

Aspect 60: The method of any one of aspects 55-60, further comprising orienting the electrode based on feedback of an accelerometer coupled to the electrode array.

Aspect 61: A method comprising:
implanting a marker under the skin of the patient, wherein the marker is indicative of a location for positioning an electrode array for providing tumor treating fields.

Aspect 62: The method of aspect 61, wherein implanting the marker comprises implanting the marker within a resection cavity.

Aspect 63: The method of aspect 61 or aspect 62, wherein the marker is positioned proximate to a surface of the skin such that the marker can be felt by touching the skin.

Aspect 64: The method of aspect 63, wherein the marker comprises a material that is configured to guide an electric field.

Aspect 65: The method of aspect 64, wherein the material is conductive.

Aspect 66: The method of aspect 64, wherein the material is dielectric.

Aspect 67: The method of any one of aspects 61-66, wherein the marker comprises a biocompatible material.

Aspect 68: The method of aspect 67, wherein the biocompatible material comprises titanium.

Aspect 69: The method of any one of aspects 61-68, wherein positioning the electrode array on the skin of the patient comprises positioning the electrode array on a back of the patient.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A system comprising:
an electrode array comprising a plurality of electrodes; and
a marker configured to be associated with skin of a patient for indicating a location on the skin for positioning the electrode array.

2. The system of claim 1, wherein the marker is configured to be implanted under the skin of the patient.

3. The system of claim 2, wherein the marker comprises a material that is configured to guide an electric field.

4. The system of claim 2, wherein the electrode array comprises:
a marker sensor that is configured to detect proximity of or alignment with the marker when the electrode array is correctly positioned on the patient; and
an indicator in communication with the marker sensor, wherein the indicator is configured to cause an indication upon the marker sensor being in proximity of or alignment with the marker.

5. The system of claim 4, wherein the indicator comprises an audible indicator, a visual indicator, or a haptic indicator.

6. The system of claim 1, wherein the marker comprises a visible marker.

7. The system of claim 6, wherein the visible marker comprises a temporary tattoo.

8. The system of claim 6, wherein the visible marker comprises indications of at least a portion of a perimeter of the electrode array.

9. The system of claim 6, wherein the electrode array defines at least one opening therethrough, wherein the visible marker comprises at least one marking that is visible through the at least one opening when the electrode array is in the location on the skin for positioning the electrode array.

10. The system of claim 9, wherein the at least one opening comprises a plurality of openings therethrough, wherein the at least one marking comprises a plurality of markings that are visible through respective openings of the plurality of openings when the electrode array is in the location on the skin for positioning the electrode array.

11. The system of claim 10, wherein the plurality of openings comprise a plurality of slots, wherein the plurality of markings comprise a plurality of elongate markings that are visible through respective slots of the plurality of slots when the electrode array is in the location on the skin for positioning the electrode array.

12. The system of claim 1, wherein the electrode array comprises an orientation sensor that is configured to detect an orientation of the electrode array.

13. The system of claim 12, wherein the orientation sensor is in communication with an orientation indicator, wherein the orientation indicator is configured to cause an indication upon the sensor being in a proper orientation.

14. The system of claim 13, wherein the orientation indicator is coupled to the electrode array.

15. The system of claim 13, wherein the orientation indicator is associated with a device that is separate from the electrode array.

16. An electrode array comprising:
a plurality of electrodes; and
a marker sensor that is configured to detect proximity of or alignment with a marker.

17. The electrode array of claim 16, further comprising an indicator in communication with the marker sensor, wherein the indicator is configured to:
cause an indication upon the marker sensor detecting proximity of or alignment with the marker.

18. A method comprising:
positioning an electrode array comprising a plurality of electrodes onto skin of a patient based on a marker associated with the skin of the patient,
wherein the marker indicates a location on the skin for positioning the electrode array.

19. The method of claim 18, wherein the marker is implanted under the skin of the patient, wherein the marker comprises a material that is configured to guide an electric field.

20. The method of claim 18, wherein the electrode array comprises:
a marker sensor that is configured to detect proximity of or alignment with the marker; and
an indicator in communication with the marker sensor, wherein the indicator is configured to cause an indication upon the marker sensor being in proximity of or alignment with the marker, wherein positioning the electrode array comprises positioning the electrode array based on the indication from the indicator.
